(19) United States
(12) Patent Application Publication
(10) Pub. No.: US 2026/0167708 A1
(43) Pub. Date: Jun. 18, 2026

(54) ANTI-TSLP ANTIBODY COMPOSITIONS AND USES THEREOF

(71) Applicant: AMGEN INC., Thousand Oaks, CA (US)

(72) Inventors: Marisa K. Joubert, Newbury Park, CA (US); Nathan H. Joh, Newbury Park, CA (US)

(21) Appl. No.: 19/123,647

(22) PCT Filed: Oct. 25, 2023
(86) PCT No.: PCT/US2023/077811

§ 371 Date: Apr. 23, 2025

Related U.S. Application Data
(60) Provisional application No. 63/380,994, filed on Oct. 26, 2022.

(51) Int. Cl. C07K 16/24; A61K 39/00; A61M 5/20

(52) U.S. Cl. CPC C07K 16/244; A61M 5/20; A61K 2039/505; A61K 2039/54; A61K 2039/545; A61M 2202/07; C07K 2317/41; C07K 2317/94

(57) ABSTRACT

The present application relates, in general, to compositions comprising anti-TSLP antibody, such as tezepelumab, and derivatives thereof having antibody quality attributes.

### CROSS-REFERENCE TO RELATED APPLICATIONS

This application claims benefit under 35 U.S.C. § 119(e) of U.S. Provisional Patent Application No. 63/380,994, filed Oct. 26, 2022, which is incorporated by reference herein in its entirety for all purposes.

### FIELD OF THE DISCLOSURE

The present application relates, in general, to compositions comprising anti-TSLP antibody, such as tezepelumab, and derivatives thereof comprising antibody quality attributes.

### BACKGROUND OF THE DISCLOSURE

Thymic stromal lymphopoietin (TSLP), an epithelial cell-derived cytokine produced in response to environmental and pro-inflammatory stimuli, leads to the activation of multiple inflammatory cells and downstream pathways (Soumelis et al. Nat Immunol 2002; 3:673-80; Allakhverdi et al. J Exp Med 2007; 204:253-8). TSLP is increased in the airways of patients with asthma and correlates with Th2 cytokine and chemokine expression (Shikotra et al. J Allergy Clin Immunol 2012; 129:104-11 e1-9) and disease severity (Ying et al. J Immunol 2005; 174:8183-90; Ying et al. J Immunol 2008; 181:2790-8). While TSLP is central to the regulation of Th2 immunity, it has been found to be implicated in inflammation and therefore may be relevant to multiple asthma phenotypes.

Tezepelumab is a human immunoglobulin G2 (IgG2) monoclonal antibody (mAb) that binds to TSLP, preventing its interaction with the TSLP receptor complex. It will be appreciated that tezepelumab is a heterotetramer comprising two heavy chains and two light chains, and comprising two binding sites to TSLP. A proof-of-concept study in patients with mild, atopic asthma, demonstrated that tezepelumab inhibited the early and late asthmatic responses and suppressed biomarkers of Th2 inflammation following inhaled allergen challenge (Gauvreau, et al. N Engl J Med 2014; 370:2102-10).

### SUMMARY

Monitoring of antibody therapeutics in a formulation over time is important to determine storage conditions that reduce any breakdown of the therapeutic and maintain the integrity of the product. The present disclosure provides a study of attributes of an anti-TSLP antibody that can change over time during manufacturing and storage, including attributes that can be beneficial or detrimental to antibody tolerability and/or potency.

In one aspect, the disclosure provides a composition comprising an anti-TSLP antibody and one or more anti-TSLP antibody derivatives, wherein the composition comprises less than 97% acidic peak species as determined by cation exchange high performance liquid chromatography (CEX-HPLC), wherein anti-TSLP antibody comprises:
(A) a light chain variable domain comprising:
(i) a light chain CDR1 amino acid sequence set out in SEQ ID NO: 3;
(ii) a light chain CDR2 amino acid sequence set out in SEQ ID NO: 4; and
(iii) a light chain CDR3 amino acid sequence set out in SEQ ID NO: 5; and
(B) a heavy chain variable domain comprising:
(i) a heavy chain CDR1 amino acid sequence set out in SEQ ID NO: 6;
(ii) a heavy chain CDR2 amino acid sequence set out in SEQ ID NO: 7 and
(iii) a heavy chain CDR3 amino acid sequence set out in SEQ ID NO: 8.

In various embodiments, the disclosure provides a composition comprising an anti-TSLP antibody and one or more derivatives thereof, wherein the composition comprises less than 97% acidic peak species as determined by cation exchange high performance liquid chromatography (CEX-HPLC), wherein the anti-TSLP antibody comprises:
(A) a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11;
iii. a sequence of amino acids encoded by a polynucleotide that hybridizes under moderately stringent conditions to the complement of a polynucleotide consisting of SEQ ID NO: 11;
wherein the light chain variable domain retains the complementary determining regions (CDRs) set out in SEQ ID NO: 3-5, and,
(B) a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO: 9;
iii. a sequence of amino acids encoded by a polynucleotide that hybridizes under moderately stringent conditions to the complement of a polynucleotide consisting of SEQ ID NO: 9;
wherein the heavy chain variable domain retains the complementary determining regions (CDRs) set out in SEQ ID NO: 6-8;
wherein the antibody or antibody derivative specifically binds to a TSLP polypeptide as set forth in amino acids 29-159 of SEQ ID NO: 2. In some embodiments, the disclosure provides a composition comprising an anti-TSLP antibody and one or more anti-TSLP antibody derivatives, wherein the composition comprises less than 97% acidic peak species as determined by cation exchange high performance liquid chromatography (CEX-HPLC), wherein anti-TSLP antibody comprises a light chain variable domain comprising the amino acid sequence set out in SEQ ID NO: 12; and a heavy chain variable domain comprising the amino acid sequence set out in et out in SEQ ID NO:10.

In various embodiments, the CEX-HPLC is cation exchange ultra-high performance liquid chromatography (CEX-UHPLC).

In various embodiments, the composition comprises (i) between 25% and 97% acidic peak species. In various embodiments, the composition comprises a 210 mg unit dose of tezepelumab. In various embodiments, the tezepelumab composition is at a dose of 420 mg and the acidic peak species are less than 48.5%. In various embodiments, the acidic peak species are selected from the group consisting of antibody fragments, partially reduced species, sialylated glycan variants, β-galactosylated glycan variants, deamidated species, disulfide isoforms B and A/B, and glycation variants.

Also provided by the disclosure is a composition comprising an anti-TSLP antibody and one or more derivatives, wherein the composition:
(i) comprises less than 64% total basic peak species as determined by cation exchange ultra-high performance liquid chromatography (CEX-HPLC); and/or
(ii) comprises no more than 5.3%, or no more than 5%, of a basic peak species that is, in order of retention time in CEX-HPLC, a third peak after the main peak, (which may also be referred to herein as a “basic peak species three”)
wherein the anti-TSLP antibody comprises
(A) a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11;
iii. a sequence of amino acids encoded by a polynucleotide that hybridizes under moderately stringent conditions to the complement of a polynucleotide consisting of SEQ ID NO: 11;
wherein the light chain variable domain retains the complementary determining regions (CDRs) set out in SEQ ID NO: 3-5, and,
(B) a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO: 9;
iii. a sequence of amino acids encoded by a polynucleotide that hybridizes under moderately stringent conditions to the complement of a polynucleotide consisting of SEQ ID NO: 9;
wherein the heavy chain variable domain retains the complementary determining regions (CDRs) set out in SEQ ID NO: 6-8;
wherein the antibody or antibody derivative specifically binds to a TSLP polypeptide as set forth in amino acids 29-159 of SEQ ID NO: 2.

In various embodiments, the composition comprises (i) between 15% and 64% total basic peak species. In various embodiments, the composition comprises (ii) no more than 5% of the basic peak species three. In various embodiments, the composition comprises (ii) no more than 5.3% of the basic peak species three. In various embodiments, the composition comprises (iii) no more than 1.5% of the basic peak species three. In various embodiments, the composition is at a 210 mg unit dose of anti-TSLP antibody. In various embodiments, the anti-TSLP antibody composition is at a unit dose of 420 mg and the total basic peak species are less than 32%. In various embodiments, the anti-TSLP antibody composition is at a unit dose of 700 mg and the total basic peak species are less than 19%. The basic peak species may comprise or consist of a total basic fraction comprising or consisting of high molecular weight species, antibody fragments, partially reduced species, heavy chain C terminal lysine and N-terminal signaling peptide, glycosylation variants (such as afucosylated, high mannose glycan, and sialylated variants), heavy chain oxidized methionine species, CDR aspartic acid isomerized species and disulfide isoform A. In various embodiments, the basic peak species are selected from the group consisting of high molecular weight species, antibody fragments, partially reduced species, heavy chain C terminal lysine and N-terminal signaling peptide, glycosylation variants (such as afucosylated, high mannose glycan, and sialylated variants), heavy chain oxidized methionine species, CDR aspartic acid isomerized species, and disulfide isoform A. In various embodiments, the anti-TSLP antibody is tezepelumab.

In various embodiments, the CEX-HPLC is cation exchange ultra-high performance liquid chromatography (CEX-UHPLC).

In various embodiments, a composition as described herein has a relative potency of at least 80%, 81%, 82%, 83%, 84%, 85%, 86%, 87%, 88%, 89%, 90% or 95% compared to a reference anti-TSLP antibody, such as a reference tezepelumab antibody. In various embodiments, the potency is determined by a receptor ligand binding assay.

In various embodiments, the receptor ligand binding assay comprises
A:
i) attaching thymic stromal lymhopoeitin (TSLP) to acceptor beads and TSLP receptor (TSLPR) to donor beads;
ii) incubating the anti-TSLP antibody or anti-TSLP antibody derivative composition with a composition comprising TSLP- and TSLPR-beads; and
iii) detecting binding of TSLP to TSLPR based on levels of luminescence produced when the TSLP acceptor beads and TSLPR donor beads are in proximity; or
B.
(i) contacting a cell line expressing TSLPR and a reporter gene with the anti-TSLP antibody or anti-TSLP antibody derivative composition, wherein the reporter gene is expressed upon binding of TSLP to TSLPR; and
ii) detecting binding of TSLP to TSLPR based on levels of luminescence produced by expression of the reporter gene.

In various embodiments, the anti-TSLP antibody comprises a heavy chain variable region amino acid sequence set out in SEQ ID NO: 10 and a light chain variable region amino acid sequence set out in SEQ ID NO: 12. In various embodiments, the anti-TSLP antibody comprises a heavy chain amino acid sequence set out in SEQ ID NO: 13 and a light chain amino acid sequence set out in SEQ ID NO: 14. In various embodiments, the anti-TSLP antibody is tezepelumab.

In various embodiments, the anti-TSLP antibody composition is obtained from a Chinese Hamster Ovary (CHO) cell line that expresses a nucleic acid encoding a heavy chain of SEQ ID NO: 10 and a nucleic acid encoding a light chain of SEQ ID NO: 12. In various embodiments, the anti-TSLP antibody is tezepelumab.

In various embodiments, the immunoglobulin, antigen binding protein or antibody is a human antibody. In various embodiments, the antibody is an IgG2 antibody. In various embodiments, the anti-TSLP antibody or derivative thereof specifically binds to a TSLP polypeptide as set forth in amino acids 29-159 of SEQ ID NO: 2. In various embodiments, both binding sites of anti-TSLP antibody or derivative thereof have identical binding to TSLP.

In various embodiments, the anti-TSLP antibody or derivative thereof binds TSLP with an affinity that is numerically no more than 10⁻⁸ M Kd.

In various embodiments, the CEX-HPLC is carried out on a 4.6 mm×50 mm, 5 μm particle size cation exchange column using a linear salt gradient at 35° C.

In various embodiments, mobile phase A consists of 20 mM sodium phosphate (74% sodium phosphate monobasic, 26% sodium phosphate dibasic) and mobile phase B consists of 20 mM sodium phosphate (74% sodium phosphate monobasic, 26% sodium phosphate dibasic), 500 mM sodium chloride. There is no pH adjustment for the mobile phase.

In various embodiments, the linear salt gradient is an increasing gradient from 5 mM to 415 mM. In various embodiments, the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min. In various embodiments, the mobile phase is applied to the column at a flow rate of 0.5 mL/min.

In various embodiments, the CEX-HPLC comprises loading 100 μg anti-TSLP antibody onto a cation exchange column. In various embodiments, the CEX-HPLC comprises loading 0.71 μL anti-TSLP antibody onto a cation exchange column.

In various embodiments, for a composition described herein,
the CEX-HPLC is carried out on a 4.6 mm×50 mm, 5 μm particle size cation exchange column using a linear salt gradient at 35° C.,
the CEX-HPLC comprises a linear salt gradient that is an increasing gradient from 5 mM to 415 mM,
wherein the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min, wherein a mobile phase A consists of 20 mM sodium phosphate (74% sodium phosphate monobasic, 26% sodium phosphate dibasic) and the mobile phase B consists of 20 mM sodium phosphate (74% sodium phosphate monobasic, 26% sodium phosphate dibasic), 500 mM sodium chloride,
wherein the mobile phase is applied to the column at a flow rate of 0.5 mL/min; and
the CEX-HPLC comprises loading 100 μg anti-TSLP antibody onto the cation exchange column in a volume of 0.71 μL.

HPLC may be run on an UHPLC instrument.

In various embodiments, for a composition described herein,
the CEX-UHPLC is carried out on a 4.6 mm×50 mm, 5 μm particle size cation exchange column using a linear salt gradient at 35° C.,
the CEX-UHPLC comprises a linear salt gradient that is an increasing gradient from 5 mM to 415 mM,
wherein the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min, wherein a mobile phase A consists of 20 mM sodium phosphate (74% sodium phosphate monobasic, 26% sodium phosphate dibasic) and the mobile phase B consists of 20 mM sodium phosphate (74% sodium phosphate monobasic, 26% sodium phosphate dibasic), 500 mM sodium chloride,
wherein the mobile phase is applied to the column at a flow rate of 0.5 mL/min; and
the CEX-UHPLC comprises loading 100 μg anti-TSLP antibody onto the cation exchange column in a volume of 0.71 μL.

In various embodiments, there is no pH adjustment for the mobile phase.

Further contemplated is a composition comprising an anti-TSLP antibody, such as tezepelumab, or derivatives thereof as described herein and a pharmaceutically acceptable carrier, excipient or diluent.

The disclosure also provides an isolated nucleic acid comprising a polynucleotide sequence encoding the light chain variable domain, the heavy chain variable domain, or both, of the anti-TSLP antibody, such as tezepelumab, or derivative thereof described herein.

The disclosure further contemplates a recombinant expression vector comprising the nucleic acid encoding the anti-TSLP antibody as described herein. Also provided is a host cell comprising the expression vector.

Further contemplated herein is a method of producing a composition comprising an anti-TSLP antibody or derivatives thereof that specifically binds to a TSLP polypeptide comprising amino acids 29-159 of SEQ ID NO: 2, comprising incubating the host cell under conditions that allow it to express the immunoglobulin, antigen binding protein, or antibody, wherein said host cell comprises (i) a recombinant expression vector encoding the light chain variable domain of the antigen binding protein of as described herein and a recombinant expression vector encoding the heavy chain variable domain of the antigen binding protein as described herein, or (ii) a recombinant expression vector encoding both the light chain variable domain and the heavy chain variable domain of the anti-TSLP antibody.

Also provided herein is a method for treating an inflammatory disease in a subject comprising administering to the subject a therapeutically effective amount of a composition comprising an anti-TSLP antibody and derivatives thereof as described herein. In various embodiments, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF). In various embodiments, the asthma is mild, moderate or severe asthma. In various embodiments, the asthma is severe asthma. In various embodiments, the asthma is eosinophilic or non-eosinophilic asthma.

In various embodiments, the method comprises administering the composition at an interval of every 2 weeks or every 4 weeks. In various embodiments, the composition is administered for a period of at least 4 months, 6 months, 9 months, 1 year or more.

In various embodiments, the antibody is an IgG2 antibody. In various embodiments, the anti-TSLP antibody is tezepelumab or tezepelumab derivatives comprising a heavy chain variable region set out in SEQ ID NO: 10 and a light chain variable region set out in SEQ ID NO: 12, and comprises one or more of the attributes described herein.

The disclosure also provides a composition comprising an anti-TSLP antibody, such as tezepelumab, and derivatives thereof as described herein for use in treating an inflammatory disease. In certain embodiments, the disclosure provides use of a composition comprising an anti-TSLP antibody, such as tezepelumab, and derivatives thereof as described herein in the preparation of a medicament for treating an inflammatory disease.

Syringes, e.g., single use or pre-filled syringes, sterile sealed containers, e.g. vials, bottle, vessel, and/or kits or packages comprising any of the foregoing antibodies or compositions, optionally with suitable instructions for use, are also contemplated. In various embodiments, the administration is via pre-filled syringe or autoinjector. In various embodiments, the auto-injector is an Ypsomed YpsoMate® device.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawings and/or claims.

### BRIEF DESCRIPTION OF THE DRAWINGS

FIG. 1 illustrates a CEX-UHPLC profile of tezepelumab drug substance.

### DETAILED DESCRIPTION

The structure of tezepelumab was elucidated from a variety of biological, biochemical, and biophysical techniques to provide an understanding of its structure and functional properties and assessment of critical quality attributes.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “inflammatory disease” refers to a medical condition involving abnormal inflammation caused by the immune system attacking the body's own cells or tissues, which may result in chronic pain, redness, swelling, stiffness, and damage to normal tissues. Inflammatory diseases include, for example, asthma, chronic peptic ulcer, tuberculosis, periodontitis, sinusitis, active hepatitis, ankylosing spondylitis, rheumatoid arthritis, chronic obstructive pulmonary disease (COPD), Crohn's disease, ulcerative colitis, osteoarthritis, atherosclerosis, systemic lupus erythematosus, atopic dermatitis, eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps, idiopathic pulmonary fibrosis (IPF), and the like. In exemplary aspects, the inflammatory disease is asthma, atopic dermatitis, COPD, eosinophilic esophagitis (EoE), nasal polyps, chronic spontaneous urticaria. In exemplary aspects, the inflammatory is asthma and, in some instances, the asthma is severe asthma, eosinophilic asthma, non-eosinophilic asthma, or low eosinophil asthma.

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response. Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of less than or equal to 300 cells/μL, or less than or equal to 250 cells/μL “Low eosinophilic” asthma refers to asthma patients having less than 250 cells/μL blood or serum. Alternatively, “low eosinophilic” asthma refers to asthma patients having less than 300 cells/μL blood or serum.

A “T helper (Th) 1 cytokine” or “Th1-specific cytokine” refers to cytokines that are expressed (intracellularly and/or secreted) by Th1 T cells, and include IFN-g, TNF-α, and IL-12.

A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-α, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds a target antigen with greater affinity than other antigens of similar sequence. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-α) and the like. In various embodiments, the antibody specifically binds the target antigen, but can cross-react with an ortholog of a closely related species, e.g., an antibody may bind human protein and also bind a closely related primate protein. In various embodiments, the immunoglobulin, antigen binding protein or fragment thereof, or antibody or fragment thereof specific for TLSP binds with a Kd that is numerically less than or equal to 10⁻⁸ M. In various embodiments, an anti-TSLP antibody described herein binds at least with an affinity (Kd) of 10⁻⁸ M, 10⁻⁹ M, 10⁻¹⁰ M, 10⁻¹¹ M, 10⁻¹² M, 10⁻¹³ M or less.

The term “antibody” refers to a tetrameric glycoprotein that consists of two heavy chains and two light chains, each comprising a variable region and a constant region. “Heavy Chains” and “Light Chains” refer to substantially full-length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies.

Antigen binding proteins include antibodies, antibody fragments and antibody-like proteins that can have changes to structure of canonical tetrameric antibodies. Antibody “variants” refer to antigen binding proteins or fragments thereof that can have structural changes in antibody sequence or function compared to a parent antibody having a known sequence. Antibody variants include variable (“V”) regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody variants or derivatives also include immunoglobulins, antigen binding proteins or fragments thereof having modifications of the N- and C-termini, which may be part of the natural processing of antibodies. The signal peptide at the N-terminus is cleaved at a specific site by signal peptidase during co-translational translocation of proteins across the ER membrane, so is not part of the mature protein sequence. However, the fidelity of this reaction is not 100%, and low level miscleavage has been observed in some antibody products. Such miscleavage can give rise to heterogeneity in the mature protein, resulting in either some level of elongation or truncation of the N-terminus of the mature protein chains (Ying et al, Immunol Lett 111: 66-8, 2007; Beck et al, Antibodies 8:18, 2019).

The antibody heavy chain of all four IgG subclasses (IgG1, IgG2, IgG3, and IgG4) displays heterogeneity at its C-terminus (Shah et al, J Pharm Sci, in press, 2022; Beck, supra). Therapeutic IgG mAbs are known to contain three C-terminal variants in their heavy chains: 1) the unprocessed C-terminal lysine (K), 2) the processed C-terminal K, and 3) C-terminal amidation (Tsubaki et al, International journal of biological macromolecules. 52:139-47, 2013). All three C-terminal variants (unprocessed C-terminal K, processed C-terminal K, and C-terminal amidation) have been detected in endogenous IgG from myeloma patients and human plasma, suggesting that they are a common modification in vivo (Shah, supra).

By way of example, a composition comprising tezepelumab may comprise N- and/or C-terminal variants of the antibody having a heavy chain amino acid sequence set out in SEQ ID NO: 13 and a light chain amino acid sequence set out in SEQ ID NO: 14. A composition comprising tezepelumab and tezepelumab derivatives may comprise N- and/or C-terminal variants of the antibody having a heavy chain amino acid sequence set out in SEQ ID NO: 13 and a light chain amino acid sequence set out in SEQ ID NO: 14, and derivatives of such N- and/or C-terminal variants.

Antibody fragments include antigen-binding portions of the antibody including, inter alia, Fab, Fab′, F(ab′)2, Fv, domain antibody (dAb), complementarity determining region (CDR) fragments, CDR-grafted antibody binding regions, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Antibody derivative” as used herein refers to antibodies, antigen binding proteins or fragments thereof comprising one or more attributes described herein, which may be characterized in terms of its chemical identity, chemical modification or structural attribute type (e.g., HMW species, fragment or isoform) and exhibits the desired biological activity.

“Valency” refers to the number of antigen binding sites on each antibody or antibody fragment that targets an epitope. A typical full length IgG molecule, or F(ab)2 is “bivalent” in that it has two identical target binding sites. A “monovalent” antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and Example 8 and US 2012/0020988 A1, Examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells (Swedin et al. Pharmacol Ther 2017; 169:13-34). TSLP signals through a heterodimeric receptor consisting of the interleukin (IL)-7 receptor alpha (IL-7Ra) chain and a common y chain-like receptor (TSLPR) (Pandey, Nat Immunol. 1:59-64, 2000; Park, J Exp Med. 192:659-669, 2000). The polynucleotide sequence of human TSLP is set out in SEQ ID NO: 1 and the polypeptide sequence is set out in SEQ ID NO: 2.

Human TSLP mRNA (Brightling et al., J Allergy Clin Immunol 2008; 121:5-10; quiz 1-2; Ortega et al. N Engl J Med 2014; 371:1198-207) and protein levels (Ortega et al., supra) are increased in the airways of asthmatic individuals compared to controls, and the magnitude of this expression correlates with disease severity (Brightling et al., supra). Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility (Ortega et al. N Engl J Med 2014; 371:1198-207; To et al. BMC Public Health 2012; 12:204). These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al, J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al., J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al., Clin Exp Allergy. 39(1):89-100, 2009). These data and other emerging evidence suggest that blocking TSLP may serve to suppress multiple biologic pathways including but not limited to those involving Th2 cytokines (IL-4/13/5).

### Antibodies

It is contemplated that antibodies or antibody derivatives or antigen binding proteins specific for TSLP as described herein are useful in the treatment of inflammatory diseases, including asthma, such as severe asthma, eosinophilic asthma, non-eosinophilic/low-eosinophilic and other forms of asthma described herein, atopic dermatitis, EoE, nasal polyps, chronic spontaneous urticaria, and COPD.

Specific binding agents such as antibodies and antibody derivatives or fragments that bind to their target antigen, e.g., TSLP, are useful in the methods and compositions of the disclosure. In one embodiment, the specific binding agent is an antibody. The antibodies may be monoclonal (MAbs). Other anti-TSLP antigen binding proteins include those generated by recombinant DNA techniques, such as the expression of recombinant plasmids containing nucleic acid sequences encoding antibody variable regions.

Monoclonal antibodies may be modified for use as therapeutics or diagnostics.

Also encompassed by the disclosure are human antibody variants and derivatives (including antibody fragments and derivatives) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Antigen binding proteins, such as monoclonal antibodies, may be produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein. Further examples of mammalian cells include immortalized cell lines available from the American Type Culture Collection (Manassas, VA), including, in addition to Chinese Hamster Ovary (CHO) cells, HeLa cells, baby hamster kidney (BHK) cells, monkey kidney cells (COS), human hepatocellular carcinoma cells (e.g., Hep G2), and human epithelial kidney 293 cells. Furthermore, cell lines or host systems can be chosen to ensure correct modification and processing of the anti-TSLP antibody or anti-TSLP antibody derivatives. Eukaryotic host cells that possess the cellular machinery for proper processing of the primary transcript, glycosylation, and phosphorylation of the gene product can be used. These include CHO, VERY, BHK, Hela, COS, MDCK, 293, 3T3, W138, BT483, Hs578T, HTB2, BT20 and T47D, NS0 (a murine myeloma cell line that does not endogenously produce any functional immunoglobulin chains), SP20, CRL7030 and HsS78Bst cells. Human cell lines developed by immortalizing human lymphocytes can also be used. The human cell line PER.C6® (Janssen; Titusville, NJ) can also be used to recombinantly produce monoclonal antibodies.

By way of example, anti-TSLP antibody and anti-TSLP antibody derivatives having molecular or clinical attributes as described herein may be obtained by selecting a cell clone that expresses the anti-TSLP antibody or a anti-TSLP antibody derivative having the attributes. Recombinant DNA methods may be used for producing such anti-TSLP antibody or derivatives. For example, DNA encoding the heavy chain and light chain of the anti-TSLP antibody or derivatives can be inserted into a suitable expression vector (or vectors, for example one vector for the heavy chain and one for the light chain), which can be transfected into a suitable host cell, such as a cell of a mammalian cell line. Suitable expression vectors are known in the art, containing, for example, a polynucleotide that encodes the anti-TSLP antibody polypeptide linked to a promoter. The expression vector may be transferred to a host cell by conventional techniques, and the transfected cells may be cultured to produce the antibodies. Optionally, the host cells may be engineered to modulate molecular attributes. For example, to modulate fucosylation, glycosylation-competent cells may be genetically modified to alter the activity of a fucosyl-transferase or a Golgi GDP-fucose transporter. By way of example, cell line engineering to modulate glycosylation is described in PCT Pub. No. WO 2015/116315.

Clones producing the anti-TSLP antibody or anti-TSLP antibody derivatives comprising the relevant molecular attributes may be selected. By way of example, established microtiter plate-based method of clone generation and growth may be performed. Hundreds of pooled, heterogeneous cells may be sorted into single-cell cultures through processes such as fluorescence-activated cell sorting (FACS) or limiting dilution. After being allowed to recover to healthy and stable populations, these clonally-derived cells may be analyzed, and select populations are chosen for further analysis. For further analysis, clone cells may be cultured in small containers, such as spin tubes, 24-well plates, or 96-deep well plates are cultured in a “small-scale cell culture” (e.g., a 10-day fed batch process). In this small-scale process, boluses of nutrients are added periodically, and different measurements of cell growth and viability are obtained. Hundreds or even thousands of these small-scale cultures may be in parallel. At the end of the culture (e.g., the tenth day), the cells are harvested for assays and analysis. Optionally, the microtiter plate-based method of clone generation and growth (e.g., subcloning) may be substituted with the use of an automated or partially automated high-throughput and high-content screening tool, such as the Berkeley Lights Beacon™ opto-electronic cell line generation and analysis system, for example. Optionally, high throughput screening methods and machine learning tools may be used to expedite the selection of clones producing the relevant molecular attributes (See, e.g., PCT Pub. No. WO 2020/223422).

In various embodiments, the anti-TSLP antibody is tezepelumab. Anti-TSLP antibody tezepelumab is described in U.S. Pat. No. 7,982,016 and U.S. patent application Ser. No. 15/951,602.

Anti-TSLP antigen binding protein (including fragments thereof) useful in the present methods comprise an anti-TSLP antibody comprising a. a light chain variable domain comprising: i. a light chain CDR1 sequence comprising the amino acid sequence set forth in SEQ ID NO: 3; ii. a light chain CDR2 sequence comprising the amino acid sequence set forth in SEQ ID NO: 4; iii. a light chain CDR3 sequence comprising the amino acid sequence set forth in SEQ ID NO: 5; and, b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO: 7, and iii. a heavy chain CDR3 sequence comprising the amino acid sequence set forth in SEQ ID NO: 8, wherein the antibody or antibody derivative specifically binds to a TSLP polypeptide as set forth in amino acids 29-159 of SEQ ID NO: 2. The anti-TSLP antigen binding protein may be tezepelumab.

Also contemplated is an antibody or antibody derivative comprising a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and, b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO: 9; iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b), wherein the antibody or antibody derivative specifically binds to a TSLP polypeptide as set forth in amino acids 29-159 of SEQ ID NO: 2.

Tezepelumab is an exemplary anti-TSLP antibody having: a. i. a light chain CDR1 sequence comprising the amino acid sequence set forth in SEQ ID NO: 3; ii. a light chain CDR2 sequence comprising the amino acid sequence set forth in SEQ ID NO: 4; iii. a light chain CDR3 sequence comprising the amino acid sequence set forth in SEQ ID NO: 5; and b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO: 7, and iii. a heavy chain CDR3 sequence comprising the amino acid sequence set forth in SEQ ID NO: 8.

Tezepelumab also comprises a light chain variable domain having the amino acid sequence set out in SEQ ID NO: 12; encoded by a polynucleotide sequence set out in SEQ ID NO: 11; and a heavy chain variable domain having the amino acid sequence set out in SEQ ID NO: 10, encoded by a polynucleotide sequence set out in SEQ ID NO: 9.

In various embodiments, the anti-TSLP antibody or antibody derivative thereof is bivalent and selected from the group consisting of a human antibody, a monoclonal antibody, a recombinant antibody, and an IgG2 antibody.

In various embodiments, the anti-TSLP antigen binding protein is selected from the group consisting of a diabody, a triabody, a tetrabody, a Fab fragment, single domain antibody, scFv, wherein the dose is adjusted such that the binding sites to be equimolar to the those dosed by bivalent antibodies.

It is contemplated that the anti-TSLP antibody or antibody derivative is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production. Tezepelumab is an IgG2 antibody. The sequence of the full-length heavy chain and light chain of tezepelumab, including the IgG2 chain, is set out in SEQ ID NOs: 13 and 14, respectively.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinities, (4) inhibit formation of high molecular weight (HMW) species, and/or (5) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Methods of Making

Anti-TSLP antibody compositions of the disclosure can be prepared by recombinantly expressing nucleic acids encoding the heavy chain and light chain in a host cell, partially purifying or purifying anti-TSLP antibody from host cell cultures or host cell lysates, and analyzing the resulting compositions for one or more of the anti-TSLP antibody derivatives detailed herein according to the methods described in more detail below. In some aspects, the method comprises rejecting a batch or lot of antibody that does not comprise acidic peak species or basic peak species within a range describe herein. The method may comprise selecting a batch or lot of antibody for further manufacturing processes when it comprises acidic peak species or basic peak species within a range described herein. For example, the method may comprise analyzing a batch of anti-TSLP antibody, e.g., tezepelumab, using cation exchange high performance liquid chromatography (CEX-HPLC) or cation exchange ultra-high performance liquid chromatography (CEX-UHPLC) as described herein, and determining an amount of acidic peak species and/or an amount of basic peak species. The method may comprise rejecting the batch when it does not comprise (i) less than 97% acidic peak species as determined by CEX-HPLC or CEX-UHPLC, (ii) less than 64% total basic peak species as determined by CEX-HPLC or CEX-UHPLC, or (iii) no more than 5%, or no more than 5.3%, of a basic peak species three that is, in order of retention time in CEX-HPLC or CEX-UHPLC, a third peak after the main peak.

For recombinant production of anti-TSLP antibody or derivatives thereof, one or more nucleic acids encoding the heavy chain (e.g. heavy chain polypeptide comprising the amino acid sequence of SEQ ID NO: 10) and light chain (e.g. light chain polypeptide comprising the amino acid sequence of SEQ ID NO: 12) is inserted into one or more expression vectors. The nucleic acid encoding the heavy chain and the nucleic acid encoding the light chain can be inserted into a single expression vector or they can be inserted into separate expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Vectors may also include one or more selectable marker genes to facilitate selection of host cells into which the vectors have been introduced. Exemplary nucleic acids encoding the heavy and light chains of tezepelumab as well as suitable signal peptide sequences and other components for expression vectors for recombinantly expressing tezepelumab are described in U.S. Pat. No. 7,982,016, which is hereby incorporated by reference in its entirety, and set out in SEQ ID NO: 9 and SEQ ID NO: 11 herein.

After the expression vector has been constructed and the one or more nucleic acid molecules encoding the heavy and light chain components of the anti-TSLP antibody or derivative thereof has been inserted into the proper site(s) of the vector or vectors, the completed vector(s) may be inserted into a suitable host cell for amplification and/or polypeptide expression. The transformation of an expression vector for anti-TSLP antibody or derivative thereof into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989).

A host cell, when cultured under appropriate conditions, synthesizes anti-TSLP antibody or derivative thereof that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurnum, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibodies from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. CHO cells are preferred host cells in some embodiments for expressing anti-TSLP antibody or derivatives thereof.

Host cells are transformed or transfected with the above-described expression vectors for production of an anti-TSLP antibody or derivatives thereof and are cultured in conventional nutrient media modified as appropriate for inducing promoters, selecting transformants, or amplifying the genes encoding the desired sequences. The host cells used to produce an anti-TSLP antibody or derivatives thereof may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinary skilled artisan.

Upon culturing the host cells, the antibody can be produced intracellularly, in the periplasmic space, or directly secreted into the medium. If the antibody is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the antibody is secreted into the culture medium, the antibody can be separated from host cells through centrifugation or microfiltration, and optionally, subsequently concentrated through ultrafiltration. Anti-TSLP antibodies or derivatives thereof can be further purified or partially purified using, for example, one or more chromatography steps, such as affinity chromatography (e.g. protein A or protein G affinity chromatography), cation exchange chromatography, anion exchange chromatography, hydroxyapatite chromatography, hydrophobic interaction chromatography, or mixed mode chromatography.

Once an anti-TSLP antibody composition is produced or obtained, the composition may be evaluated for the presence and amount of one or more anti-TSLP antibody derivatives described herein that may be isolated in a CEX-HPLC acidic or basic peak, including isomerization derivatives (including isomerization intermediates thereof), deamidation derivatives (including deamidation intermediates thereof), oxidation derivatives, glycosylation derivatives, disulfide isoform derivatives and size derivatives (e.g. HMW species or fragments). Accordingly, the present disclosure includes methods for assessing the quality of an anti-TSLP antibody composition, comprising obtaining an anti-TSLP antibody composition that contains anti-TSLP antibody and one or more anti-TSLP antibody derivatives; measuring the amount of one or more anti-TSLP antibody derivatives in the composition, or a collection of all the derivatives; comparing the measured amount of the one or more anti-TSLP antibody derivatives to a pre-determined reference criterion; and preparing a pharmaceutical formulation or pharmaceutical product of the anti-TSLP antibody composition if the comparison indicates that the pre-determined reference criterion is met. In some embodiments, the methods comprise measuring one or more acidic and/or basic peak fractions from a CEX-HPLC column. In some embodiments, the methods comprise identifying anti-TSLP antibody derivatives in the CEX-HPLC analysis, comprising identifying one or more of: isomerization derivatives (including isomerization intermediates thereof) in the composition, deamidation derivatives (including deamidation intermediates thereof) in the composition, oxidation derivatives in the composition, glycosylation derivatives in the composition, disulfide isoform derivatives in the composition, HMW species in the composition, glycation derivatives, partially reduced species, N-terminal and C-terminal variants and/or fragments in the composition. In certain embodiments, all listed derivatives are identified in an anti-TSLP antibody composition. In various embodiments, the anti-TSLP antibody is tezepelumab.

The pre-determined reference criterion for each anti-TSLP antibody derivative can be a threshold amount or range of amounts of the derivative that do not significantly affect the potency and/or tolerability of the anti-TSLP antibody composition, e.g., for safety purposes during administration or for inhibiting ligand-induced activation of the TSLP receptor. For instance, the pre-determined reference criterion for each anti-TSLP antibody derivative can be any of the limits or ranges disclosed herein for each of the derivatives as anti-TSLP antibody compositions with these limits/ranges of the derivatives had comparable potency and/or tolerability to anti-TSLP antibody compositions evaluated in clinical trials and shown to have clinical efficacy. It will be appreciated that pre-determined reference criteria described herein may be specified prior to the commencement of a method as described herein.

In certain embodiments of the methods, if the measured amount of the anti-TSLP antibody derivative in the composition meets the pre-determined reference criterion, then the anti-TSLP antibody composition can be classified as acceptable and progressed to the next step in the manufacturing or distribution process, such as for example, by preparing a pharmaceutical formulation of the composition (e.g. by combining with one or more excipients or diluents); by preparing a pharmaceutical product of the composition (e.g., by filling into vials, syringes, autoinjectors, or other containers or delivery devices); packaging the composition with instructions for use, diluents, and/or delivery devices; or releasing the composition for commercial sale or shipping to distributors. In some embodiments of the methods, a pharmaceutical formulation of the anti-TSLP antibody composition is prepared if the measured amount of the anti-TSLP antibody derivative in the composition meets the pre-determined reference criterion. In other embodiments of the methods, a pharmaceutical product of the anti-TSLP antibody composition is prepared if the measured amount of the anti-TSLP antibody derivative in the composition meets the pre-determined reference criterion. Methods of preparing pharmaceutical formulations and pharmaceutical products of anti-TSLP antibody compositions are described in more detail below. If the measured amount of the anti-TSLP antibody derivative in the composition does not meet the pre-determined reference criterion, then, in some embodiments of the methods, the anti-TSLP antibody composition can be classified as unacceptable and discarded, destroyed, or subject to additional manufacturing steps, such as additional purification to remove or reduce the amount of the anti-TSLP antibody derivative in the composition such that the pre-determined reference criterion is met.

In one embodiment, the methods for assessing the quality of an anti-TSLP antibody composition comprise obtaining an anti-TSLP antibody composition that contains anti-TSLP antibody and anti-TSLP antibody CEX-HPLC peaks; measuring the amount of the acidic and basic peaks in the composition; comparing the measured amount of the acidic and basic peaks to a pre-determined reference criterion; and preparing a pharmaceutical formulation or pharmaceutical product of the anti-TSLP antibody composition if the comparison indicates that the pre-determined reference criterion is met. The pre-determined reference criterion for the amount of acidic and basic peaks in an anti-TSLP antibody composition can be (i) less than 64% total basic peak species as determined CEX-HPLC; and/or (ii) no more than 5.3%, or no more than 5%, of a basic peak species three that is, in order of retention time in CEX-HPLC, a third peak after the main peak. In various embodiments, the anti-TSLP antibody is tezepelumab.

In one embodiment, the anti-TSLP antibody composition comprises (i) between 15% and 64% total basic peak species, between 20% and 60% total basic peak species, or between 25% and 50% total basic peak species. In some embodiments, the anti-TSLP antibody composition comprises 15%, 20%, 25%, 30%, 35%, 40%, 45%, 50%, 60% or 64% total basic peak species, between 20% and 60% total basic peak species, or between 25% and 50% total basic peak species. In other embodiments, the composition comprises no more than 5.3%, 5%, 4%, 3%, 2% or 1.5% of the basic peak species three. In various embodiments, the anti-TSLP antibody is tezepelumab.

The pre-determined reference criterion for the amount of acidic and basic peaks in an anti-TSLP antibody composition may comprise less than 97% acid peak species as determined CEX-HPLC. In various embodiments, the anti-TSLP antibody is tezepelumab.

In one embodiment, the anti-TSLP antibody composition comprises (i) between 25% and 97% acidic peak species, between 25% and 80% acidic peak species, between 25% and 60% acidic peak species, between 35% and 80% acidic peak species, or between 45% and 75% total basic peak species. In some embodiments, the anti-TSLP antibody composition comprises 25%, 30%, 35%, 40%, 45%, 50%, 55%, 60%, 65%, 70%, 75%, 80%, 85%, 90%, 95% or 97% acidic peak species. In various embodiments, the anti-TSLP antibody is tezepelumab.

In certain embodiments of the methods of the disclosure, the methods comprise:
(a) obtaining an anti-TSLP antibody composition that contains anti-TSLP antibody and one or more derivatives;
(b) evaluating the anti-TSLP antibody composition by performing one or more of the following:
   (i) measuring the amount of species in a CEX-HPLC acidic and/or basic peak fraction
(c) preparing a pharmaceutical formulation or pharmaceutical product of the anti-TSLP antibody composition if the comparison or comparisons in step (b) indicate that the pre-determined reference criterion/criteria are met.

In various embodiments, the anti-TSLP antibody derivatives comprise isomerization derivatives, deamidation derivatives, oxidation derivatives, glycosylation, disulfide isoform derivatives, glycation derivatives, HMW species, partially reduced species, N-terminal and C-terminal variants, fragments or combinations thereof.

It is contemplated that the CEX-HPLC is carried out on a 4.6 mm×50 mm, 5 μm particle size cation exchange column using a linear salt gradient at 35° C. The mobile phase A consists of 20 mM sodium phosphate (74% sodium phosphate monobasic, 26% sodium phosphate dibasic) and mobile phase B consists of 20 mM sodium phosphate (74% sodium phosphate monobasic, 26% sodium phosphate dibasic), 500 mM sodium chloride. There is no pH adjustment for the mobile phase.

In certain embodiments, the linear salt gradient is an increasing gradient from 5 mM to 415 mM. An exemplary linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min. In various embodiments, the mobile phase is applied to the column at a flow rate of 0.5 mL/min.

In various embodiments, the CEX-HPLC comprises loading 100 μg anti-TSLP antibody onto a cation exchange column. In various embodiments, the CEX-HPLC comprises loading 0.71 μL anti-TSLP antibody onto a cation exchange column.

In various embodiments, for an anti-TSLP antibody composition described herein,
the CEX-HPLC is carried out on a 4.6 mm×50 mm, 5 μm particle size cation exchange column using a linear salt gradient at 35° C.,
the CEX-HPLC comprises a linear salt gradient that is an increasing gradient from 5 mM to 415 mM,
wherein the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min, wherein a mobile phase A consists of 20 mM sodium phosphate (74% sodium phosphate monobasic, 26% sodium phosphate dibasic) and the mobile phase B consists of 20 mM sodium phosphate (74% sodium phosphate monobasic, 26% sodium phosphate dibasic), 500 mM sodium chloride, and there is no pH adjustment for the mobile phase,
wherein the mobile phase is applied to the column at a flow rate of 0.5 mL/min; and
the CEX-HPLC comprises loading 100 μg anti-TSLP antibody onto the cation exchange column in a volume of 0.71 μL.

It is contemplated that the CEX-HPLC is CEX-UHPLC. The CEX-UHPLC may be carried out on an UHPLC instrument (e.g., Waters Corporation, Milford, MA)

In some embodiments, the composition is at a 210 mg unit dose of anti-TSLP antibody. In another embodiment, the anti-TSLP antibody composition is at a unit dose of 420 mg and the total basic peak species are less than 32%. In certain embodiments, the anti-TSLP antibody composition is at a unit dose of 700 mg and the total basic peak species are less than 19%. In various embodiments, the anti-TSLP antibody is tezepelumab.

### Identification of Attributes Affecting Drug Quality

In order to determine attributes that contribute to protein binding and activity, anti-TSLP antibody, such as tezepelumab, as described herein is placed in a condition that leads to a change in its structure, for example, a change in the structure of an amino acid of the therapeutic protein, leading to the formation of a derivative of the therapeutic protein. In exemplary aspects, the changed structure of an amino acid is referred to as an “attribute” and may be characterized in terms of its chemical identity or attribute type and location within the amino acid sequence of the antigen binding protein, e.g., the position of the amino acid on which the attribute is present. For example, asparagine and glutamine residues are susceptible to deamidation. A deamidated asparagine at position 10 of a protein amino acid sequence is an example of an attribute. A list of exemplary attribute types for particular amino acids is provided in Table A. As such, a “structure” as used herein can comprise, consist essentially of, or consisting of an attribute type listed in Table A, or a combination of two or more attribute types listed in Table A. It will be understood that attributes are examples of structures, and unless stated otherwise, wherever a “structure” is mentioned herein, an attribute is contemplated as an example of the structure. For example, high molecular weight species (HMW) and fragments are also examples of attributes.

TABLE A

| Exemplary Attribute Type | Amino acid residue |
|---|---|
| deamidation | Asn, Gln |
| glycation, hydroxylysine | Lys |
| glycosylation | Asn |
| cyclization | N-terminal Gln, N-terminal Glu |
| oxidation | Met, Trp, His |
| isomerization | Asp |
| fragmentation/clipping | Asp/Pro |

As an immunoglobulin or fragment thereof, antibody or antigen binding protein, such as tezepelumab, comprises multiple amino acids, an anti-TSLP antibody or antigen binding protein described herein may have more than one attribute (e.g., more than one amino acid having a changed structure) and may be described in terms of its attribute profile. As used herein, the term “attribute profile” refers to a listing of an antigen binding protein's attributes. In various instances, the attribute profile provides the chemical identity or attribute type, e.g., deamidation, optionally, relative to the native structure of the therapeutic protein. In various instances, the attribute profile provides the location of the attribute, e.g., the position of the amino acid on which the attribute is present. An attribute profile in some aspects, provides a description of all attributes present on the antigen binding protein. In other aspects, an attribute profile provides a description of a subset of attributes present on the protein. For example, an attribute profile may provide only those attributes that are present in a particular portion of the protein, e.g., the constant region, the variable region, the CDRs (such as the three light chain CDRs and the three heavy chain CDRs). A species of a therapeutic protein such as an antibody or antigen binding protein is characterized by the attribute(s) present on the protein. A species of an antibody or antigen binding protein may differ from another species of the same protein by having a different attribute profile. When two therapeutic proteins have differing attribute profiles, the therapeutic proteins represent two different species or derivatives of the therapeutic protein. When two therapeutic proteins have identical attribute profiles, the therapeutic proteins are considered as the same species or derivative of the therapeutic protein.

The levels of attributes critical to the drug quality, or critical quality attributes (CQAs), may be explicitly defined by the product purity specifications. These specifications are typically subject to extensive regulatory reviews. In some embodiments, a specification may set the permissible levels of one or more molecular attributes in the manufacture of a biological therapy.

In various instances, the immunoglobulin, antibody or antigen binding protein is placed in a condition that leads to a change in its structure, e.g., formation of one or more attributes, and the change in structure may alter the affinity of the immunoglobulin, antibody or antigen binding protein for its target. In various aspects, the immunoglobulin, antibody or antigen binding protein is placed in a condition that leads to a change in its structure, e.g., formation of one or more attributes, and the change in structure reduces the affinity of the antigen binding protein for its target. The reduced affinity in some aspects leads to a partial or total loss of the ability of the immunoglobulin, antibody or antigen binding protein to interact with (e.g., bind to) a target. In various instances, the partial or total loss of the ability of the immunoglobulin, antibody or antigen binding protein to interact with (e.g., bind to) a target ultimately reduces the antigen binding protein's efficacy. In alternative instances, the immunoglobulin, antibody or antigen binding protein is placed in a condition that leads to a change in its structure, e.g., formation of one or more attributes, and the change in structure does not alter the affinity of the immunoglobulin, antibody or antigen binding protein for its target. In various aspects, the change in structure does not reduce the affinity of the protein for its target.

In various aspects, a composition herein comprises a population of species or derivatives of the immunoglobulin, antigen binding protein or fragment thereof, or antibody or fragment thereof. In various instances, the population is a homogenous population of the immunoglobulin, antigen binding protein or fragment thereof, or antibody or fragment thereof, optionally, each of the proteins present in the composition sample are the same species or derivative. In various instances, the population is a heterogeneous population comprising at least two different species or derivatives of the immunoglobulin, antigen binding protein or fragment thereof, or antibody or fragment thereof having an attribute described herein. In various aspects, the heterogeneous population comprises at least 2, at least 3, at least 4, at least 5, at least 6, at least 7 or more different species or derivative of the immunoglobulin, antigen binding protein or fragment thereof, or antibody or fragment thereof. Optionally, the heterogeneous population comprises more than 7, more than 8, more than 9, more than 10, more than 20, more than 30, more than 40, more than 50 different species or derivatives of the protein. Each species or derivative of the population in some aspects has a unique attribute profile. In exemplary instances, the species of the immunoglobulin, antigen binding protein or fragment thereof, or antibody or fragment thereof are the only proteins present in the composition. In some aspects, the composition comprises (i) the population immunoglobulin, antigen binding protein or fragment thereof, or antibody or fragment thereof immunoglobulin, antigen binding protein or fragment thereof, or antibody or fragment thereof and (ii) a pharmaceutically-acceptable carrier, diluent, excipient, or a combination thereof. In some embodiments, at least 80%, 85%, 90%, 95%, or 99% of immunoglobulin, antigen binding protein or fragment thereof, or antibody or fragment thereof of the heterogeneous population comprises an attribute as described herein. In some embodiments, no more than 20%, 15%, 10%, 5%, or 1% of immunoglobulin, antigen binding protein or fragment thereof, or antibody or fragment thereof of the heterogeneous population comprises an attribute as described herein.

### Separation

In various embodiments of the method of identifying attributes of an immunoglobulin, anti-TSLP antibody or antigen binding protein (e.g., tezepelumab) or target that affect an interaction between the immunoglobulin, antibody or antigen binding protein and the target, for each of the unbound fraction and bound fraction, the method comprises identifying and quantifying the abundance of each attribute present on a species or derivative of the immunoglobulin, antibody or antigen binding protein or target, wherein, when the abundance of an attribute in the unbound fraction is greater than the abundance of the attribute in the bound fraction, the attribute negatively affects the interaction between the immunoglobulin, antibody or antigen binding protein and the target. In various aspects, the method comprises using a mass spectrometer to identify and quantify the abundance of each attribute of the species of the immunoglobulin, antibody or antigen binding protein or target in each of the unbound fraction and bound fraction.

In various embodiments of the method of determining an effect of a known attribute present on a species of an immunoglobulin, antibody or antigen binding protein or target on an interaction between the antigen binding protein and the target, the method comprises for each of the unbound fraction and bound fraction, quantifying the abundance of the known attribute, wherein, when the abundance of the known attribute in the unbound fraction is greater than the abundance of the known attribute in the bound fraction, the known attribute has a negative effect on the interaction between the immunoglobulin, antibody or antigen binding protein and the target. In various aspects, the method comprises using a mass spectrometer to quantify the abundance of the known attribute in each of the unbound fraction and bound fraction.

Stability refers to resistance to chemical modifications of amino acid residues and biophysical protein modifications, such as formation of HMW species during stress conditions which may occur during manufacturing, storage and/or additional or alternative stress conditions. For methods and immunoglobulins, antigen binding proteins, and fragments thereof of embodiments described herein, “stability” and/or “HMW” species, may be determined using size exclusion chromatography (SEC).

It will be appreciated that “affinity” or “binding” may be determined by surface plasmon resonance (SPR), bio-layer interferometry, or also by SEC binding affinity experiments as described herein. Unless stated otherwise herein or necessitated otherwise by scientific context, “affinity” will be understood to refer to affinity as measured by SPR. Kd value may be measured by SPR using a biosensor system such as a BIAcore® system. The analysis with the BIAcore® system may comprise analyzing the binding and dissociation of an antigen (e.g., TSLP) from chips with immobilized molecules (e.g., anti-TSLP immunoglobulin, antigen binding protein, or fragment thereof as described herein) on their surface. Binding complexes with Kd<10⁻⁶ M can be detected using SPR. In various embodiments, the SPR may be carried out at 20° C., 25° C., 30° C. or 37° C.

### Compositions

It will be appreciated that numbering of the residues in tezepelumab is based on the heavy chain and light chain variable sequences set out in SEQ ID NOs: 10 and 12, respectively, as well as the full-length antibody heavy chain and light chain set out in SEQ ID NOs: 13 and 14, respectively.

In various embodiments, provided is a composition comprising an anti-TSLP antibody and one or more anti-TSLP antibody derivatives, each comprising: a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ IDNO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, wherein the derivatives comprise at least one of: isomerization derivatives, deamidation derivatives, oxidation derivatives, glycosylation derivatives, HMW species, partially reduced species, N-terminal and C-terminal variants, fragments, disulfide isoform derivatives, or combinations thereof, which may be encompassed in the CEX acidic or basic peaks of the antibody compositions. In various embodiments, the composition comprises an anti-TSLP antibody and one or more anti-TSLP antibody derivatives, each comprising a heavy chain amino acid sequence set out in SEQ ID NO: 10 and a light chain amino acid sequence set out in SEQ ID NO: 12.

Antibodies comprise charge variants due to post-translational modification and degradation events during storage. Acidic species refer to antibody variants with lower apparent pl compared to the non-variant antibody, and basic species are antibody variants with higher apparent pl, e.g., as measured by isoelectric focusing. Acidic species and basic species are characterized by comparing retention times during chromatographic analysis relative to the main peak. Acidic species are variants that elute prior to the main peak from cation exchange chromatography (CEX) or after the main peak from anion exchange chromatography (AEX). Basic species are variants that elute after the main peak from CEX or prior to the main peak from AEX.

In various embodiments, for any anti-TSLP antibody or anti-TSLP antibody derivative composition described herein, the anti-TSLP antibody or tezepelumab derivative has a relative potency of at least 75%, 80%, 81%, 82%, 83%, 84%, 85%, 86%, 87%, 88%, 89%, 90%, 91%, 92%, 93%, 94%, 95%, 96%, 97%, 98% or 99% compared to a reference anti-TSLP antibody.

For example, the reference standard anti-TSLP antibody may be from a lot that has been confirmed to inhibit binding of TSLP to TSLPR. The reference standard anti-TSLP antibody may be an anti-TSLP antibody having the same amino acid sequences as those of the antibody of the composition described herein. In various embodiments, the anti-TSLP antibody is tezepelumab.

In various embodiments, for any for any anti-TSLP antibody or derivative composition described herein, including a pharmaceutical composition thereof, the relative potency comprises or consists of relative inhibition of the ability of anti-TSLP antibody to inhibit the binding of TSLP to TSLPR.

In various embodiments, for any anti-TSLP antibody or anti-TSLP antibody derivative composition described herein, the relative potency is as measured by receptor ligand binding assay comprising: attaching TSLP to acceptor beads and TSLPR to donor beads; incubating the anti-TSLP antibody or anti-TSLP antibody derivative antibody composition with a composition comprising TSLP- and TSLPR-beads; and detecting the binding of TSLP to TSLPR based on levels of luminescence produced when the TSLP acceptor beads and TSLPR donor beads are in proximity.

In various embodiments, for any anti-TSLP antibody or anti-TSLP antibody derivative composition described herein, the relative potency is as measured by cell-based reporter gene assay comprising: contacting a cell line expressing TSLPR and a reporter gene, wherein the reporter gene is expressed upon binding of TSLP to TSLPR with the anti-TSLP antibody or anti-TSLP antibody derivative antibody composition; and detecting the binding of TSLP to TSLPR based on levels of luminescence produced by the expression of the reporter gene.

In some embodiments, the composition is part of a formulation described herein. In some embodiments, the composition is a drug substance used to produce a formulation as described herein.

### Methods of Administration

In one aspect, methods of the present disclosure include a step of administering a therapeutic anti-TSLP antibody or antibody derivative described herein, optionally in a pharmaceutically acceptable carrier or excipient. In certain embodiments, the pharmaceutical composition is a sterile composition.

Contemplated herein are methods for treating an inflammatory disease, condition or disorder, such as asthma, chronic obstructive pulmonary disease (COPD), atopic dermatitis, eosinophilic esophagitis (EoE), nasal polyps, chronic spontaneous urticaria, Ig-driven disease, IgA nephropathy, lupus nephritis, eosinophilic gastritis, chronic sinusitis with or without nasal polyps and idiopathic pulmonary fibrosis (IPF) with an anti-TSLP antibody or antigen binding protein or fragments thereof as described herein. In various embodiments, the disease, condition or disorder is asthma, including severe asthma, eosinophilic or non-eosinophilic asthma and low eosinophil asthma.

Asthma is a chronic inflammatory disorder of the airways. Each year, asthma accounts for an estimated 1.1 million outpatient visits, 1.6 million emergency room visits, 444,000 hospitalizations (Defrances et al, 2008) Available at: The Centers for Disease Control website, www.cdc.gov/nchs/data/nhsr/nhsr005.pdf, and 3,500 deaths in the U.S. In susceptible individuals, asthmatic inflammation causes recurrent episodes of wheezing, breathlessness, chest tightness, and cough. The etiology of asthma is thought to be multi-factorial, influenced by both genetic environmental mechanisms (To et al., BMC Public Health 2012; 12:204; Chung et al. Eur Respir J 2014; 43:343-73), with environmental allergens an important cause (Chung et al., supra; Pavord ID, et al., NPJ Prim Care Respir Med 2017; 27:17). The majority of cases arise when a person becomes hypersensitive to allergens (atopy). Atopy is characterized by an increase in Th2 cells and Th2 cytokine expression and IgE production. Approximately 10 million patients in the United States are thought to have allergy-induced asthma. Despite the available therapeutic options, asthma continues to be a major health problem. Worldwide, asthma currently affects approximately 300 million people; by 2020, asthma is expected to affect 400 million people (Partridge, Eur Resp Rev. 16:67-72, 2007).

Allergen inhalation by atopic asthmatics induces some of the manifestations of asthma, including reversible airflow obstruction, airway hyperresponsiveness, and eosinophilic and basophilic airway inflammation. Allergen inhalation challenge has become the predominant model of asthma in many species (Bates et al., Am J Physiol Lung Cell Mol Physiol. 297(3):L401-10, 2009; Diamant et al., J Allergy Clin Immunol. 132(5):1045-1055, 2013.)

Different asthma subtypes that are refractory to steroid treatment have been identified. Eosinophils are important inflammatory cells in allergic asthma that is characteristically mediated by Th2-type CD4+ T cells. Neutrophilic airway inflammation is associated with corticosteroid treatment in severe asthma and can be mediated by Th1- or Th17-type T cells (Mishra et al., Dis. Model. Mech. 6:877-888, 2013).

Measures of diagnosis and assessment of asthma include the following: Airway inflammation evaluated using a standardized single-breath Fraction of Exhaled Nitric Oxide (FeNO)(American Thoracic Society; ATS, Am J Respir Crit Care Med. 171(8):912-30, 2005) test. Spirometry is performed according to ATS/European Respiratory Society (ERS) guidelines (Miller et al, Eur Respir J. 26(1):153-61, 2005). Post-bronchodilator (Post-BD) spirometry testing is assessed after the subject has performed pre-BD spirometry. Maximal bronchodilation is induced using a SABA such as albuterol (90 μg metered dose) or salbutamol (100 μg metered dose) or equivalent with a spacer device for a maximum of 8 total puffs (Sorkness et al, J Appl Physiol. 104(2):394-403, 2008). The highest pre- and post-BD FEV₁ obtained after 4, 6, or 8 puffs is used to determine reversibility and for analysis. Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV₁ (Juniper et al, October 1999). The ACQ-6 is a shortened version of the ACQ that omits the FEV₁ measurement from the original ACQ score. The mean ACQ score is the mean of the responses. Mean scores of 0.75 indicate well-controlled asthma, scores between 0.75 and s 1.5 indicate partly-controlled asthma, and a score >1.5 indicates uncontrolled asthma (Juniper et al, Respir Med. 100(4):616-21, 2006). Individual changes of at least 0.5 are considered to be clinically meaningful (Juniper et al, Respir Med. 99(5):553-8, 2005). The Asthma Quality of Life Questionnaire, Standardized (AQLQ[S])+12 (AQLQ(S)+12) is a 32-item questionnaire that measures the HRQoL experienced by asthma patients (Juniper et al, Chest. 115(5):1265-70, May 1999). The Asthma Daily Diary is also used for assessment.

Related US Patent Publication US-2018-0296669 (incorporated herein by reference) discloses that treatment with an anti-TSLP antibody is effective at reducing asthma symptoms in a no eosinophil/low eosinophil population as it is in a high eosinophil population. Also contemplated is a method of reducing the frequency of asthma exacerbation in a subject.

Also contemplated herein are methods of treating asthma in a subject having a Th2 high asthma profile or a Th2 low asthma profile. It is contemplated that a TSLP antagonist that inhibits binding of the TSLP protein to its receptor complex will effectively treat a low eosinophil asthma population as the antibody described herein. Similarly, it is contemplated that a TSLP antagonist that inhibits binding of TSLP to its receptor complex will be effective in treating Th2 low asthma populations. Also contemplated are methods for treating chronic obstructive pulmonary disease (COPD) in a subject comprising administering an anti-TSLP antibody or antibody derivative or antigen binding protein described herein. It is contemplated that the subject to be treated is human. The subject may be an adult, an adolescent or a child.

Therapeutic antibody (or antibody derivative) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic immunoglobulin, antibody or antigen binding protein, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody derivative is administered in a dose range of about 70 mg to about 280 mg per daily dose. For example, the dose may be given in about 70 mg, 210 mg or 280 mg. In various embodiments, the anti-TSLP antibody or antibody derivative may be administered at a dose of 70, 80, 90, 100, 110, 120, 130, 140, 150, 160, 170, 180, 190, 200, 210, 220, 230, 240, 250, 260, 270 or 280 mg per dose. These concentrations may be administered as a single dosage form or as multiple doses. The above doses are given every two weeks or every four weeks. In various embodiments, the anti-TSLP antibody or antibody derivative is administered at a single dose of 70 mg every two weeks or every four weeks. In various embodiments, the anti-TSLP antibody or antibody derivative is administered at a single dose of 210 mg every two weeks or every four weeks. In various embodiments, the anti-TSLP antibody or antibody derivative is administered at a single dose of 280 mg every two weeks or every four weeks.

For antibody derivatives, the amount of antibody derivative should be such that the number of TSLP binding sites that are in the dose have an equimolar number of TSLP binding sites to canonical bivalent antibody described above.

It is contemplated that the anti-TSLP antibody or antibody derivative is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody derivative is contemplated to decrease eosinophils in blood, sputum, broncheoalveolar fluid, or lungs of the subject. It is also contemplated that the administration shifts cell counts in the subject from a Th2 high population to a Th2 low population. It is further contemplated that administration of the anti-TSLP antibody improves one or more measures of asthma in a subject selected from the group consisting of forced expiratory volume (FEV), FEV1 reversibility, forced vital capacity (FVC), FeNO, Asthma Control Questionnaire-6 score and AQLQ(S)+12 score.

Improvement in asthma may be measured as one or more of the following: reduction in AER (annualized exacerbation rate), reduction in hospitalizations/severe exacerbations for asthma, change from baseline (increase) in time to first asthma exacerbation (following onset of treatment with anti-TSLP antibody), decrease relative to placebo in proportion of subjects with one or more asthma exacerbations or severe exacerbations over the course of treatment, e.g., 52 weeks, change from baseline (increase) in FEV1 and FVC (pre-broncholdilator and post-bronchodilator), change from baseline (decrease) in blood or sputum eosinophils (or lung eosinophils if biopsy or BAL fluid obtained), change from baseline (decrease) in FeNO, change from baseline (decrease) in IgE, improvement in asthma symptoms and control as measured by PROs including ACQ and variants, AQLQ and variants, SGRQ, and asthma symptom diaries, change (decrease) in use of rescue medications, decrease in use of systemic corticosteroids, decrease in Th2/Th1 cell ratio in blood. Most/all of these measures should be in total population and subpopulations including high and low eosinophils (in certain embodiments, greater than or equal to 250 is high; less than 250 is low; in certain embodiments, greater than or equal to 300 is high; less than 300 is low), allergic and non-allergic, Th2 high and low, periostin high and low (compared to median value), and FeNO hi and low (greater than or equal to 24 or less than 24).

Also contemplated in the present disclosure is the administration of multiple agents, such as an antibody composition in conjunction with a second agent as described herein, including but not limited to an anti-inflammatory agent or asthma therapy.

However, it is contemplated that, in various embodiments, the administration reduces frequency of or levels of co-administered therapy in the subject. Exemplary co-administered therapies include, but are not limited to, inhaled corticosteroids (ICS), long-acting β32 agonist (LABA), leukotriene receptor antagonists [LTRA], long-acting anti-muscarinics [LAMA], cromones, short-acting β32 agonist (SABA), and theophylline or oral corticosteroids. In various embodiments, the administration eliminates the need for corticosteroid therapy.

### Formulations

In some embodiments, the disclosure contemplates use of pharmaceutical compositions comprising a therapeutically effective amount of an anti-TSLP antibody or antibody derivative together with a pharmaceutically acceptable diluent, carrier, solubilizer, emulsifier, preservative, and/or adjuvant. In addition, the disclosure provides methods of treating a subject by administering such pharmaceutical composition. In various embodiments, for any pharmaceutical composition described herein, the anti-TSLP antibody is authorized for administration to a human subject by a government regulatory agency.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute thereof.

The formulation components are present preferably in concentrations that are acceptable to the site of administration. In certain embodiments, buffers are used to maintain the composition at physiological pH or at a slightly lower pH, typically within a pH range of from about 4.5 to about 8. Including about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody derivative is in a formulation containing acetate, and one or more of proline, sucrose, polysorbate 20 or polysorbate 80. In various embodiments, the formulation comprises 5-50 mM acetate, less than or equal to 3% (w/v) proline, 0.015% (w/v)±0.005% (w/v) polysorbate 20 or polysorbate 80, at pH between 4.9 and 6.0. Optionally, the antibody or antibody derivative is at a concentration of between about 100 and about 150 mg/ml. The formulation may be stored at −20° to −70° C. Exemplary anti-TSLP formulations comprising these excipients are described in International Application No. PCT/US2021/018561, herein incorporated by reference.

In alternative embodiments, the anti-TSLP antibody or antibody derivative is in a formulation containing a surfactant, and at least one basic amino acid or a salt thereof. In exemplary instances, the basic amino acid is arginine or histidine. In various embodiments, the salt is arginine glutamate or histidine glutamate, optionally in a concentration of from 10 to 200 mM. Optionally, the formulation further comprises proline. In alternative embodiments, the anti-TSLP antibody or antibody derivative is in a formulation containing a surfactant, and calcium or a salt thereof. In various embodiments, the salt is calcium glutamate, optionally in a concentration from 15 mM to about 150 mM. Optionally, the formulation further comprises proline. In various embodiments, the surfactant is polysorbate 20 or polysorbate 80 or a mixture thereof. Optionally, the antibody or antibody derivative is at a concentration of greater than about 110 mg/ml, or greater than about 140 mg/ml. Exemplary anti-TSLP formulations comprising these excipients are described in International Patent Application No. PCT/US2021/017880, herein incorporated by reference.

When parenteral administration is contemplated, the therapeutic compositions for use may be provided in the form of a pyrogen-free, parenterally acceptable aqueous solution comprising the desired anti-TSLP antibody or derivative thereof in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody. In various embodiments, the administration may be via pre-filled syringe or autoinjector. In various embodiments, the auto-injector is an Ypsomed YpsoMate®. In various embodiments, the auto-injector is disclosed in WO 2018/226565, WO 2019/094138, WO 2019/178151, WO 20120/072577, WO2020/081479, WO 2020/081480, PCT/US20/70590, PCT/US20/70591, PCT/US20/53180, PCT/US20/53179, PCT/US20/53178, or PCT/US20/53176.

### Kits

As an additional aspect, the disclosure includes kits which comprise one or more compounds or compositions packaged in a manner which facilitates their use to practice methods of the disclosure. In one embodiment, such a kit includes a compound or composition described herein, packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the antibody composition.

Additional aspects and details of the disclosure will be apparent from the following examples, which are intended to be illustrative rather than limiting.

### EXAMPLES

### Example 1—Identification of Tezepelumab Attributes

Tezepelumab is a full-length, human monoclonal antibody of the IgG2 subclass produced in Chinese Hamster Ovary (CHO) cells. It consists of 2 heavy chains (HC) and 2 light chains (LC) of the lambda subclass. The heavy and light chains are covalently linked through disulfide bonds. Biochemical, biophysical, and biological characterization of tezepelumab was conducted to provide a comprehensive understanding of its structural and functional properties and to enable an assessment of antibody attributes that may affect binding and potency.

### Materials and Methods

AMG 157 and labile residues potentially impacting binding: Amino acid sequence of AMG157 as sequence A5 (and as chains H5, L5) and also several other TSLP-binding antibodies were previously described in U.S. Pat. No. 7,982,016 B2.

Molecular mass of the antibody with A2GOF/A2GOF glycosylation (C6500 H9998 O2068 N1734 S52) is 147189.4 Da, including heavy chain N-terminal pyroglutamate and C-terminal K removed. TSLP contained 74% monomeric, 23% dimeric and 3% tetrameric species.

CEX-UHPLC: Samples of tezepelumab drug substance were loaded onto an analytical CEX-UHPLC column (BioPro SP-F, 5 μm particle size, 4.6 mm×50 mm, YMC America, Inc.). The mobile phase A consists of 20 mM sodium phosphate (74% sodium phosphate monobasic, 26% sodium phosphate dibasic) and mobile phase B consists of 20 mM sodium phosphate (74% sodium phosphate monobasic, 26% sodium phosphate dibasic), 500 mM sodium chloride. There is no pH adjustment for the mobile phases. Proteins were separated using a linear salt gradient generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min. The eluent was monitored by UV absorbance at 280 nm. The column was operated at 35° C. and the mobile phase was applied to the column at a flow rate of 0.5 mL/min. The CEX-HPLC comprises loading 100 μg tezepelumab onto the cation exchange column in a volume of 0.71 μL.

Potency: Potency of the tezepelumab compositions comprising attributes described herein was observed by a receptor-ligand binding bioassay and/or a reporter gene cell-based bioassay.

Receptor-Ligand Binding Assay: This assay provides a proximal measure of tezepelumab activity and directly reflects the molecular mechanism of action of tezepelumab, which is to bind TSLP and prevent it from binding to the TSLP receptor (TSLPR). This method provides a quantitative measure of the ability of tezepelumab to inhibit the binding of TSLP to TSLPR. Tezepelumab binds to the recombinant TSLP-His ligand (TSLP-His) and inhibits it from binding to biotinylated TSLP Receptor (TSLPR). The potency assay is a bead-based Amplified Luminescent Proximity Homogeneous Assay (Alpha) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives as well as nickel chelate. The donor beads bind to biotinylated TSLPR through interaction between streptavidin and biotin, and the acceptor beads bind to histidine tagged TSLP due to the interaction between nickel chelate and histidine. When TSLP-His and biotinylated TSLPR bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence, which is measured in a plate reader equipped with AlphaScreen® signal detection capabilities. Tezepelumab binds to TSLP-His and prevents it from binding to biotinylated TSLPR, thereby decreasing the luminescence output in a dose dependent manner. The test sample activity is determined by comparing the test sample response to the response obtained for the Reference Standard. It will be appreciated that the Receptor-Ligand Binding Assay described in this paragraph is a suitable assay for determining the capability of a composition to inhibit binding of biotinylated TSLPR immobilized on a donor bead to TSLP-His immobilized on an acceptor bead.

Cell-Based Reporter Gene Bioassay: Human Thymic Stromal Lymphopoietin (TSLP) protein binding to human TSLP receptor (TSLPR) complex expressed on the surface of stable murine BaF cells induces Stat 5 activation and cell proliferation. This method utilizes the murine BaF/hu HTR cell line that were co-transfected with plasmids encoding the Stat luciferase reporter gene and blasticidin-resistant gene. When Stat/BaF/HTR cells are incubated with recombinant human TSLP, signal transduction occurs following binding to the TSLPR, resulting in the increase of luciferase activity. Tezepelumab antagonizes TSLP induced activity of the TSLPR, thus inhibiting TSLP mediated luciferase response. This method measures the dose dependent inhibitory effect of Reference Standard and test samples on Stat/BaF/HTR cells stimulated with TSLP. Following incubation with TSLP and tezepelumab, the cells are treated with a reagent containing a detergent (for cell lysis) and luciferin, a substrate for luciferase. The reaction of luciferase with luciferin results in luminescence that is measured in a luminometer. Production of luciferase in reporter cells in response to TSLP stimulation is quantified by luminescence reading after addition of luciferase substrate. The degree of inhibition of TSLP induced activation of luciferase reporter activity is proportional to the amount of tezepelumab. Test sample biological activity is determined by comparing the test sample response to the Reference Standard. It will be appreciated that the Cell-Based Reporter Gene Assay described in this paragraph is a suitable assay for determining the capability of a composition to inhibit binding of TSLPR expressed on the surface of a Stat/BaF/HTR cell encoding a Stat luciferase reporter gene, the expression of which is indicative of binding of TSLP to TSLPR.

### Results

Biochemical characterization of tezepelumab identified modified tezepelumab antibodies that could be isolated from tezepelumab preparations and after storage of drug substance including isomerization derivatives, deamidation derivatives, oxidation derivatives, high molecular weight species, fragmented species, partially reduced species, high mannose glycan derivatives, N-terminal and C-terminal variants or disulfide isoform derivatives. These attributes as a whole when isolated in CEX acidic and basic peaks were assessed for their potential impact on potency and tolerability of tezepelumab.

Cation-exchange UHPLC separates proteins based on differences in their surface charges. CEX acidic and basic species of tezepelumab in clinical pharmaceutical compositions after different storage durations at 5° C. were determined by CEX-UHPLC.

The charged variants of tezepelumab are separated on a cation-exchange column with elution using an increasing salt gradient as described above. The eluent is monitored by UV absorbance. The charged variant distribution is evaluated by determining the peak area of each variant as a percentage of the total peak area.

The CEX acidic peak contains antibody fragments, partially reduced species, sialylated glycan variants, β-galactosylated glycan variants, deamidated species, and disulfide isoforms B and A/B, and glycation variants. The CEX basic peak contains high molecular weight species, antibody fragments, partially reduced species, heavy chain C terminal lysine and N-terminal signaling peptide, glycosylation variants (such as afucosylated, high mannose glycan, and sialylated variants), heavy chain oxidized methionine species, CDR aspartic acid isomerized species, and disulfide isoform A.

Effects on potency were assessed based on amounts of tezepelumab administered to patients via 2 different routes. Patients received either a dose of 700 mg tezepelumab intravenously (Gavreau et al., NEJM 370; 2102-2101, 2014), or received a dose of 210 mg tezepelumab subcutaneously.

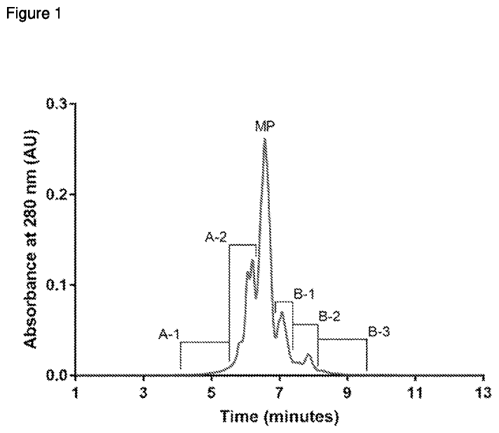

The charge variants of tezepelumab were isolated (Acidic peaks fraction A-1 and A-2; Basic peaks fractions B-1, B-2, and B-3) (FIG. 1) and assessed for potency during product characterization (Table 1 and Table 2). The isolated acidic fractions and basic fractions 1 and 2 did not show a meaningful change in potency.

TABLE 1
Potency Determination of CEX-UHPLC Fractions
and Tezepelumab Drug Substance (DS)

| | Receptor-ligand | Cell-based Reporter |
|---|---|---|
| Sample | Binding Assay | Gene Bioassay |
| Description | % Relative Potency | % Relative Potency |
| A-1 | 95 | 94 |
| A-2 | 106 | 99 |
| MP | 110 | 104 |
| B-1 | 109 | 103 |
| B-2 | 106 | 94 |
| B-3 | 95 | 82 |
| DS | 115 | 100 |

TABLE 2
Potency of Tezepelumab Drug Substance (DS) and Basic Fraction 3 (B-3)

| | | | | Cell-based |
|---|---|---|---|---|
| | Total Basic | Basic Fraction 3 | Receptor-ligand | Reporter Gene |
| DS Sample | Peaks | (B-3) | Binding Assay | Bioassay |
| Incubation Time | % Peak Area | % Peak Area | % Relative Potency | % Relative Potency |
| 0 day | 20 | 1.16 | 107 | 107 |
| 3 days | 21.4 | 1.48 | N/A | N/A |
| 7 days | 22.49 | 1.97 | N/A | N/A |
| 14 days | 24.42 | 2.77 | N/A | N/A |
| 24 days | 26.06 | 3.59 | N/A | N/A |
| 38 days | 27.17 | 5.28 | 102 | 90 |

To enrich degradation products in charge variant fractions, the stressed tezepelumab material (40C over a time course of up to four weeks) was fractionated by CEX-UHPLC. The biological activity of the isolated stressed basic fractions were evaluated by the receptor-ligand binding assay and the cell-based reporter gene bioassay (Table 3).

TABLE 3
Relative potency of CEX-UHPLC basic
fractions for stressed material

% Relative Potency

| | Receptor-ligand | Cell-based reporter |
|---|---|---|
| Sample | binding assay | gene bioassay |
| Basic Fraction B-1 | 101 | 94 |
| Basic Fraction B-2 | 93 | 86 |
| Basic Fraction B-3 | N/A | 59 |

No detectable potency change was observed for basic fraction 1 and 2. A significant change in potency was observed for basic fraction 3. The relative potency for basic fraction 3 was 59% for the cell-based reporter gene bioassay and results failed parallelism acceptance criteria in the receptor-ligand binding assay, indicating the species present are sufficiently different in activity than the untreated Reference Standard to which it was compared. However, the basic peak 3 is a relatively small part in the overall CEX-UHPLC profile (1.4% in DS) and it corresponds to only approximately 7% of the total basic peaks group in the clinical-grade tezepelumab pharmaceutical compositions tested. Due to this, the decreased potency of basic peak 3 is not expected to provide any significant impact to overall product potency, as well as potency for the combined basic peaks. For example, decreased potency of basic fraction 3 only contributes a 2.4% change in overall sample potency, i.e., the basic peak 3 percentage is 6.58% at 4 weeks of 40 C, the basic 3 peak purity is up to 90%, and the potency drop is about −41% (41%×90%×6.578%=2.4%).

It was calculated that pharmaceutical compositions comprising tezepelumab for clinical administration comprised up to 203.6 mg CEX acidic species and up to 135.4 mg CEX basic species at the time of clinical administration via intravenous (IV) injections, which corresponds to 29% CEX acidic species and 19% CEX basic species in a 700 mg dose of IV injections of tezepelumab. Thus, this level of acidic or basic species in drug is safe in patients. Extrapolating the total mg calculations based on available dose, 203.6 mg CEX acidic species and 135.4 CEX basic species respectively corresponds to 97% and 64% in 210 mg dose of IV injections of tezepelumab. 121.9 mg CEX acidic species and 81.1 mg CEX basic species respectively corresponds to 29% and 19% in 420 mg dose of SubQ injections of tezepelumab. Table 4 illustrates the allowable level of CEX peak species in the different dose administrations of tezepelumab composition.

TABLE 4

| Ab dose | CEX Acidic | CEX Basic | | % CEX Acidic | % CEX Basic |
|---|---|---|---|---|---|
| 700 mg | 203.6 mg | 135.4 | mg | 29% | 19% |
| 420 mg | 121.9 mg | 81.1 | mg | 29% | 19% |
| Projected | Possible | Possible | | Possible | Possible |
| Ab dose | CEX Acidic | CEX Basic | | % CEX Acidic | % CEX Basic |
| 210 mg | 203.6 | 135.4 | mg | 97% | 64% |
| 210 mg | 121.9 | 81.1 | mg | 58% | 39% |

| | | | | |
|---|---|---|---|---|
| 420 mg | 203.6 | 135.4 | 48.5% | 32% |

Adverse Events (AE) for clinical subjects given IV injections included: respiratory tract infection, myalgia, headache, oropharyngeal pain, dermatitis contact and asthma. AE for clinical subjects given SubQ injections included: headache, injection site reaction, upper respiratory tract infection, myalgia, back pain and dermatitis. No AE was observed to correlate with exposures to the CEX acidic or basic species in either IV- or SubQ-injected subjects. Accordingly, it was contemplated that CEX acidic species or CEX basic species can be tolerated in tezepelumab pharmaceutical compositions that remain clinically suitable.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain many equivalents to the specific embodiments of the invention described herein. Such equivalents are intended to be encompassed by the following claims.

What is claimed:

1. A composition comprising an anti-TSLP antibody and one or more anti-TSLP antibody derivatives, wherein the composition comprises less than 97% acidic peak species as determined by cation exchange ultra-high performance liquid chromatography (CEX-UHPLC), wherein anti-TSLP antibody comprises:
I. (A) a light chain variable domain comprising:
(i) a light chain CDR1 amino acid sequence set out in SEQ ID NO: 3;
(ii) a light chain CDR2 amino acid sequence set out in SEQ ID NO: 4; and
(iii) a light chain CDR3 amino acid sequence set out in SEQ ID NO: 5; and
(B) a heavy chain variable domain comprising:
(i) a heavy chain CDR1 amino acid sequence set out in SEQ ID NO: 6;
(ii) a heavy chain CDR2 amino acid sequence set out in SEQ ID NO: 7 and
(iii) a heavy chain CDR3 amino acid sequence set out in SEQ ID NO: 8;
II. (A) a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11;
iii. a sequence of amino acids encoded by a polynucleotide that hybridizes under moderately stringent conditions to the complement of a polynucleotide consisting of SEQ ID NO: 11;
wherein the light chain variable domain retains the complementary determining regions (CDRs) set out in SEQ ID NO: 3-5, and,
(B) a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO: 9;
iii. a sequence of amino acids encoded by a polynucleotide that hybridizes under moderately stringent conditions to the complement of a polynucleotide consisting of SEQ ID NO: 9;
wherein the heavy chain variable domain retains the complementary determining regions (CDRs) set out in SEQ ID NO: 6-8;
wherein the antibody or antibody derivative specifically binds to a TSLP polypeptide as set forth in amino acids 29-159 of SEQ ID NO: 2; or
III. a light chain variable domain comprising the amino acid sequence set out in SEQ ID NO: 12; and a heavy chain variable domain comprising the amino acid sequence set out in et out in SEQ ID NO: 10.

2. The composition of claim 1 comprising (i) between 25% and 97% acidic peak species.

3. The composition of claim 1 or 2, wherein the composition comprises a 210 mg unit dose of anti-TSLP antibody.

4. The composition of claim 1 or 2, wherein the anti-TSLP antibody composition is at a dose of 420 mg and the acidic peak species are less than 48.5%.

5. The composition of any one of claims 1 to 4, wherein the acidic peak species are selected from the group consisting of antibody fragments, partially reduced species, sialylated glycan variants, β-galactosylated glycan variants, deamidated species, disulfide isoforms B and A/B, and glycation variants.

6. The composition of any one of claims 1 to 5, wherein the composition has a relative potency of at least 80%, 81%, 82%, 83%, 84%, 85%, 86%, 87%, 88%, 89%, 90% or 95% compared to a reference anti-TSLP antibody.

7. The composition of claim 6, wherein the potency is determined by a receptor ligand binding assay.

8. A composition comprising anti-TSLP antibody and one or more anti-TSLP antibody derivatives, wherein the composition comprises
(i) less than 64% basic peak species as determined by cation exchange ultra-high performance liquid chromatography (CEX-HPLC), or
(ii) comprises no more than 5.3%, or no more than 5%, of a basic peak species three that is, in order of retention time in CEX-HPLC, a third peak after the main peak
wherein anti-TSLP antibody comprises:
I. (A) a light chain variable domain comprising:
(i) a light chain CDR1 amino acid sequence set out in SEQ ID NO: 3;
(ii) a light chain CDR2 amino acid sequence set out in SEQ ID NO: 4; and
(iii) a light chain CDR3 amino acid sequence set out in SEQ ID NO: 5; and
(B) a heavy chain variable domain comprising:
(i) a heavy chain CDR1 amino acid sequence set out in SEQ ID NO: 6;
(ii) a heavy chain CDR2 amino acid sequence set out in SEQ ID NO: 7 and
(iii) a heavy chain CDR3 amino acid sequence set out in SEQ ID NO: 8; or
II. (A) a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11;
iii. a sequence of amino acids encoded by a polynucleotide that hybridizes under moderately stringent conditions to the complement of a polynucleotide consisting of SEQ ID NO:11;
wherein the light chain variable domain retains the complementary determining regions (CDRs) set out in SEQ ID NO: 3-5, and,
(B) a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO: 9;
iii. a sequence of amino acids encoded by a polynucleotide that hybridizes under moderately stringent conditions to the complement of a polynucleotide consisting of SEQ ID NO: 9;
wherein the heavy chain variable domain retains the complementary determining regions (CDRs) set out in SEQ ID NO: 6-8;
wherein the antibody or antibody derivative specifically binds to a TSLP polypeptide as set forth in amino acids 29-159 of SEQ ID NO: 2.

9. The composition of claim 8 comprising (i) between 15% and 64% basic peak species.

10. The composition of claim 8 or 9 comprising (ii) no more than 5.3%, or no more than 5%, of the basic peak species three.

11. The composition of claim 8 or 9 comprising (ii) no more than 1.5% of the basic peak species three.

12. The composition of any one of claims 8 to 11, wherein the composition is at a 210 mg unit dose of anti-TSLP antibody.

13. The composition of any one of claims 8 to 11, wherein the anti-TSLP antibody composition is at a unit dose of 420 mg and the total basic peak species are less than 32%.

14. The composition of any one of claims 8 to 11, wherein the anti-TSLP antibody composition is at a unit dose of 700 mg and the total basic peak species are less than 19%.

15. The composition of any one of claims 8 to 14, wherein the basic peak species are selected from the group consisting of high molecular weight species, antibody fragments, partially reduced species, heavy chain C terminal lysine and N-terminal signaling peptide, glycosylation variants, heavy chain oxidized methionine species, CDR aspartic acid isomerized species, and Disulfide isoform A.

16. The composition of any one of claims 8 to 15, wherein the composition has a relative potency of at least 80%, 81%, 82%, 83%, 84%, 85%, 86%, 87%, 88%, 89%, 90% or 95% compared to a reference anti-TSLP antibody.

17. The composition of claim 16, wherein the potency is determined by a receptor ligand binding assay.

18. The composition of claim 7 or 17, wherein the receptor ligand binding assay comprises
A:
i) attaching thymic stromal lymhopoeitin (TSLP) to acceptor beads and TSLP receptor (TSLPR) to donor beads;
ii) incubating the anti-TSLP antibody or anti-TSLP antibody derivative antibody composition with a composition comprising TSLP- and TSLPR-beads; and
iii) detecting binding of TSLP to TSLPR based on levels of luminescence produced when the TSLP acceptor beads and TSLPR donor beads are in proximity;
or
B.
(i) contacting a cell line expressing TSLPR and a reporter gene with the anti-TSLP antibody or anti-TSLP antibody derivative antibody composition, wherein the reporter gene is expressed upon binding of TSLP to TSLPR; and
ii) detecting binding of TSLP to TSLPR based on levels of luminescence produced by expression of the reporter gene.

19. The composition of any one of claims 1 to 18, wherein the anti-TSLP antibody comprises a heavy chain variable region amino acid sequence set out in SEQ ID NO: 10 and a light chain variable region amino acid sequence set out in SEQ ID NO: 12.

20. The composition of any one of claims 1 to 19, wherein the anti-TSLP antibody comprises a heavy chain amino acid sequence set out in SEQ ID NO: 13 and a light chain amino acid sequence set out in SEQ ID NO: 14.

21. The composition of any one of claims 1 to 20, wherein the CEX-HPLC is carried out on a 4.6 mm×50 mm, 5 μm particle size cation exchange column using a linear salt gradient at 35° C.

22. The composition of claim 21, wherein mobile phase A consists of 20 mM sodium phosphate (74% sodium phosphate monobasic, 26% sodium phosphate dibasic) and mobile phase B consists of 20 mM sodium phosphate (74% sodium phosphate monobasic, 26% sodium phosphate dibasic), 500 mM sodium chloride.

23. The composition of claim 21 or 22, wherein the linear salt gradient is an increasing gradient from 5 mM to 415 mM.

24. The composition of any one of claims 21 to 23, wherein the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min.

25. The composition of any one of claims 21 to 24, wherein the mobile phase is applied to the column at a flow rate of 0.5 mL/min.

26. The composition of any one of claims 21 to 25, wherein the CEX-HPLC comprises loading 100 μg anti-TSLP antibody onto a cation exchange column.

27. The composition of claim 26, wherein the CEX-HPLC comprises loading 0.71 μL anti-TSLP antibody onto a cation exchange column.

28. The composition of any one of claims 1 to 27, wherein:
the CEX-HPLC is carried out on a 4.6 mm×50 mm, 5 μm particle size cation exchange column using a linear salt gradient at 35° C.,
the CEX-HPLC comprises a linear salt gradient that is an increasing gradient from 5 mM to 415 mM,
wherein the linear salt gradient is generated with 1% to 8.4% mobile phase B from 0 min to 6 min, to 34% mobile phase B at 14 min, to 83% mobile phase B at 14.5 min to 16 min, and to 1% mobile phase B at 16.5 min to 23 min, wherein the mobile phase A consists of 20 mM sodium phosphate (74% Sodium phosphate monobasic, 26% Sodium phosphate dibasic) and the mobile phase B consists of 20 mM sodium phosphate (74% Sodium phosphate monobasic, 26% Sodium phosphate dibasic), 500 mM sodium chloride,
wherein the mobile phase is applied to the column at a flow rate of 0.5 mL/min; and
the CEX-HPLC comprises loading 100 μg anti-TSLP antibody onto the cation exchange column in a volume of 0.71 μL.

29. The composition of any one of claims 22 to 28, wherein there is no pH adjustment for the mobile phases.

30. The composition of any one of claims 1 to 29, wherein the anti-TSLP antibody is tezepelumab.

31. A pharmaceutical formulation comprising the composition of any one of claims 1 to 30 and one or more pharmaceutically acceptable excipients.

32. A method for treating an inflammatory disease in a subject comprising administering to the subject a therapeutically effective amount of a composition of any one of claims 1 to 30 or the pharmaceutical formulation of claim 31.

33. The method of claim 32, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

34. The method of claim 32 or 33, comprising administering the composition at an interval of every 2 weeks or every 4 weeks.

35. The method of any one of claims 32 to 34, wherein the composition is administered for a period of at least 4 months, 6 months, 9 months, 1 year or more.

36. The method of any one of claims 32 to 35, wherein the asthma is severe asthma.

37. The method of any one of claims 32 to 36, wherein the asthma is eosinophilic or non-eosinophilic asthma.

38. The method of any one of claims 32 to 37, wherein the administration is via pre-filled syringe or auto-injector.

39. The method of claim 37, wherein the auto-injector is an Ypsomed YpsoMate® device.

40. An anti-TSLP antibody composition of any one of claims 1 to 30 or a pharmaceutical composition of claim 31 for use in treating an inflammatory disease in a subject.

41. The composition of claim 40, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

42. Use of an anti-TSLP antibody composition of any one of claims 1 to 30 or a pharmaceutical composition of claim 31 in the preparation of a medicament for treating an inflammatory disease in a subject.

43. The composition or use of any one of claims 40 to 42, wherein the administration is via pre-filled syringe or autoinjector.

44. The composition or use of claim 43, wherein the auto-injector is an Ypsomed YpsoMate® device.

45. The composition or use of any one of claims 40 to 44, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).


SEQUENCE LISTING (14 sequences)

SEQ ID NO: 1 - DNA, 673 bp, Homo sapiens
  source 1..673: mol_type = other DNA; note = human TSLP polynucleotide (CDS 200-676)
ctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo sapiens
  source 1..159: note = (human TSLP polypeptide)
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, synthetic construct
  REGION 1..11: note = Light chain CDR1 amino acid sequence
GGNNLGSKSVH

SEQ ID NO: 4 - protein, 7 aa, synthetic construct
  REGION 1..7: note = light chain CDR2 amino acid sequence
DDSDRPS

SEQ ID NO: 5 - protein, 11 aa, synthetic construct
  REGION 1..11: note = Light chain CDR3 amino acid sequence
QVWDSSSDHVV

SEQ ID NO: 6 - protein, 5 aa, synthetic construct
  REGION 1..5: note = Heavy chain CDR1 amino acid sequence
TYGMH

SEQ ID NO: 7 - protein, 16 aa, synthetic construct
  REGION 1..16: note = Heavy chain CDR2 amino acid sequence
VIWYDGSNKHYADSVK

SEQ ID NO: 8 - protein, 13 aa, synthetic construct
  REGION 1..13: note = Heavy chain CDR3 amino acid sequence
APQWELVHEAFDI

SEQ ID NO: 9 - DNA, 366 bp, synthetic construct
  misc_feature 1..366: note = Heavy chain variable region polynucleotide sequence
  source 1..366: mol_type = other DNA
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, synthetic construct
  REGION 1..122: note = Heavy chain variable region
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, synthetic construct
  misc_feature 1..325: note = Light chain variable region polynucleotide sequence
  source 1..325: mol_type = other DNA
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 107 aa, synthetic construct
  REGION 1..107: note = Light chain variable region
SYVLTQPPSVSVAPGQTARITCGGNNLGSKSVHWYQQKPGQAPVLVVYDDSDRPSWIPERFGSNSGNTATLTISRGEAGDEADYYCQVWDSSSDHVVFGGGTKLTVL

SEQ ID NO: 13 - protein, 444 aa, synthetic construct
  REGION 1..444: note = Full heavy chain amino acid sequence
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFKKLTVDKSRWQQGNVFSCSVMHEALHNHYTQKSLSLSPGK

SEQ ID NO: 14 - protein, 214 aa, synthetic construct
  REGION 1..214: note = Full light chain amino acid sequence
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS